(19) United States
(12) Patent Application Publication
(10) Pub. No.: US 2017/0166457 A1
(43) Pub. Date: Jun. 15, 2017

(54) APPARATUS FOR PURIFYING LIQUID BY ULTRAVIOLET LIGHT IRRADIATION

(71) Applicant: NESTEC S.A., Vevey (CH)

(72) Inventors: Celine Rimbault, Vittel (FR); Renaud Sublet, Vittel (FR)

(21) Appl. No.: 15/327,110

(22) PCT Filed: Jul. 9, 2015
(86) PCT No.: PCT/EP2015/065745

§ 371 Date: Jan. 18, 2017

(30) Foreign Application Priority Data
Jul. 18, 2014 (FR) 14177693.0

(51) Int. Cl. C02F 1/32; A23L 3/28; A61L 2/10; A23L 2/50

(52) U.S. Cl. CPC C02F 1/325; A23L 2/50; A23L 3/28; A61L 2/10; A23V 2002/00; C02F 2201/3222; C02F 2201/3227; C02F 2201/3228; C02F 2303/04; C02F 2307/10

(57) ABSTRACT

An apparatus for purifying liquid comprises a substantially tubular irradiation chamber (100) and a plurality of UV-LEDs (110) projecting ultraviolet radiation into it so as to irradiate the flow (106) of liquid being passed through it, where each of the UV-LEDs (110) is disposed upon the irradiation chamber (100) so that it is illuminated by at least one other of the plurality of UV-LEDs (110).

### TECHNICAL FIELD

The invention relates to an apparatus for purifying water by ultraviolet light irradiation, as well as a dispensing apparatus comprising it.

### BACKGROUND OF THE INVENTION

The present invention relates generally to an apparatus for purifying water, as well as to a beverage dispenser comprising it.

One of the most essential tasks in purifying liquids such as water for drinking is disinfection, so as to ensure that any pathogenic microorganisms (e.g. bacteria, viruses, and protozoans) present in the water cannot cause illness in anyone who drinks it. It is known to perform this disinfection by the process of ultraviolet (UV) irradiation, where a volume of water being treated is bombarded with high-energy radiation in the form of UV light. The UV light damages the DNA and RNA of the pathogenic microorganisms, destroying their ability to reproduce and effectively neutralizing their ability to cause disease.

Since such systems use light to disinfect, their effectiveness is reduced on liquid which is not naturally clear or which has not been filtered to remove suspended solids. The scope of “purification,” for the purposes of this document, should thus be understood as encompassing the disinfection of liquid in which turbidity is minimal.

Traditional UV liquid purification systems have employed gas-discharge lamps as UV sources, in particular mercury-vapor lamps. Recently, it has become more and more common to employ ultraviolet light-emitting diodes (UV-LEDs) as a source of ultraviolet light for irradiation. UV-LEDs have numerous advantageous aspects which makes them appealing for use in an ultraviolet liquid purification system, notably their compact size, robustness, and lack of toxic components such as the mercury vapor found in conventional lamps. The solid-state nature of UV-LEDs also enables them to be switched on and off instantly, a further advantage relative to conventional gas-discharge lamps.

It should be noted that, in this document, the term “ultraviolet light-emitting diode” is abbreviated to “UV-LED,” and that any incidence of the latter term should not be interpreted otherwise.

However, unlike traditional gas-discharge lamps, UV-LEDs tend to emit UV radiation in a conical pattern with much less diffusion of the UV light than occurs with traditional gas-discharge lamps. Configuring a system to use UV-LEDs will thus present a certain amount of difficulty, in that the emission pattern of UV-LEDs makes it much more difficult to properly illuminate the entire volume of the irradiation chamber and achieve full irradiation of the liquid therein, reducing the maximum flow rate of liquid through the irradiation chamber.

There may thus be created so-called “dead zones” within the irradiation device which receive no significant ultraviolet irradiation. This, in turn, obligates the user to reduce the flow of liquid through the irradiation device, so that the entire volume of the flow is irradiated to a sufficient degree.

There are certain systems known in the prior art which attempt to resolve this problem. The document KR 2010-0093259 describes a system where arrays of UV-LEDs are disposed in tubes which extend through the irradiation chamber; this achieves sterilization of the water flowing through the irradiation chamber, but this system requires large numbers of UV-LEDs to be effective which makes it expensive to build and to operate. The document WO 2012/078476 discloses a series of baffle-like reflectors which project from the sides of the irradiation chamber into the flow of liquid and reflect the UV light into all parts of the irradiation chamber. Similarly, the document KR 2012-003719 discloses a sterilizing apparatus where a rod-shaped light guide projects into an irradiation chamber and diffuses UV light therein from a source disposed outside the chamber. These devices successfully direct the UV light into all parts of the irradiation chamber, but their projecting nature disrupts the flow of liquid, and their surfaces may become fouled with mineral and/or biological accretions, reducing the effectiveness of the apparatus and increasing the maintenance burden upon their users.

It is thus an object of the invention to provide a water purification apparatus which resolves at least some of the foregoing problems.

### SUMMARY OF THE INVENTION

According, therefore, to a first aspect, the invention is directed towards an apparatus for purifying liquid, comprising a substantially tubular irradiation chamber adapted to conduct a flow of liquid therethrough, and a plurality of UV-LEDs disposed upon and configured to project ultraviolet radiation into said irradiation chamber and thereby irradiate said flow of liquid.

According to the invention, the plurality of UV-LEDs is configured such that each of said UV-LEDs is directly illuminated by the ultraviolet irradiation emitted by at least one other of said UV-LEDs.

In this way, the volume of the dead zones within the irradiation chamber will be reduced or even eliminated. More specifically, since each UV-LED emits ultraviolet irradiation in a conical pattern, disposing any particular UV-LED within the conical illumination pattern of at least one other UV-LED means that the volume near, but not within, the illumination pattern of that UV-LED will be irradiated.

Moreover, disposing the UV-LEDs in this fashion will maximize the volume of the irradiation chamber that is irradiated for any particular number of UV-LEDs. A liquid purification apparatus configured according to this aspect can therefore realize a maximum output for any given level of power consumption or vice-versa.

Preferably, the plurality of UV-LEDs are distributed along the length of the irradiation chamber with a substantially uniform linear spacing.

This is advantageous in that it maximizes the length of the portion of the irradiation chamber where the liquid therein is being irradiated. As sterilization effectiveness is partially a function of the irradiation time, an apparatus so configured will extend the amount of time any particular unit volume of liquid flowing through the irradiation chamber will be irradiated, thereby increasing the effectiveness of the liquid purification apparatus. In this way, the flow rate through the apparatus may be maximized without increasing its dimensions, number of UV-LEDs, or power consumption thereof.

Preferably, the plurality of UV-LEDs are distributed along the perimeter of the irradiation chamber with a substantially uniform angular spacing about a longitudinal axis of said irradiation chamber.

This is advantageous in that the angle at which the ultraviolet irradiation is directed into the volume of liquid will change as it flows through the irradiation chamber. This yields a thorough irradiation throughout the volume of the liquid, without necessitating the flow of liquid locally churn, swirl, or otherwise flow in directions not parallel to the overall direction of flow. The overall efficiency and effectiveness of the apparatus are thereby improved.

According to a preferred embodiment, each of said UV-LEDs is disposed upon the irradiation chamber directly opposite another of said UV-LEDs, thereby defining a plurality of UV-LED pairs.

This is particularly advantageous, in that the area surrounding each of the UV-LEDs will be irradiated with the strongest possible ultraviolet illumination of the other UV-LED. Furthermore, the region of the irradiation chamber directly between them will be irradiated by both UV-LEDs in the pair. The thoroughness of the purification of the liquid is thereby maximized.

Preferably, the UV-LED pairs are distributed along the length of the irradiation chamber with a substantially uniform linear spacing, and along the perimeter of said irradiation chamber with a substantially uniform angular spacing about a longitudinal axis of said chamber.

In this way, the irradiation chamber will also realize the advantages as described above in relation to the other embodiments of the invention.

In a practical embodiment, the distance along a wall of the irradiation chamber between any two adjacent UV-LEDs is less than or equal to twice the width of the irradiation chamber multiplied by the tangent of one-half the angle of emission of the UV-LEDs.

In this way, the illumination of each of the UV-LEDs is performed by at least one adjacent UV-LED. The reliability of the apparatus is thereby maximized, since as at least some of the UV-LEDs will be illuminated by multiple other UV-LEDs, the failure of a single UV-LED is less likely to result in an insufficient irradiation of the flow of liquid.

Preferably, the irradiation chamber has a substantially constant cross-section.

This is advantageous in that since the geometric relations between the UV-LEDs, the flow of liquid, and the irradiation chamber are constant over the length of the irradiation chamber, the irradiation will be of a substantially constant intensity. A substantially constant cross-section is also easier and less expensive to manufacture, such as by extrusion or other commonly-known techniques.

Preferably the cross-section is substantially circular.

This is particularly advantageous in that the cross-section of the irradiation chamber is symmetric and free from flat surfaces and sharp corners which might disrupt the flow of the liquid through it.

In a practical embodiment, the UV-LEDs have an angle of emission equal to or greater than 90°.

This is advantageous in that with a wider angle of emission the UV-LEDs may be placed on the irradiation chamber further apart from each other while still realizing the requisite co-illumination. The construction of the irradiation chamber is thus simplified, and the apparatus comprising it may be constructed at a lower cost.

Preferably, the angle of emission is between 110° and 130° inclusive, and preferably 120°.

An angle of emission in such a range is desirable in that it will create a broad cone of ultraviolet illumination within the irradiation chamber. This further ensures the elimination of dead zones within the volume of the irradiation chamber. UV-LEDs with emission angles around 120° are also commonly available in commercial quantities and power outputs.

In a practical embodiment, at least part of an interior surface of the irradiation chamber is substantially reflective to ultraviolet irradiation.

This ensures that the ultraviolet light emitted by the UV-LEDs is evenly distributed about the irradiation chamber, bringing the volume of any dead zones down to an absolute minimum. The effectiveness of the irradiation chamber is thereby maximized.

Preferably, the interior surface of the irradiation chamber is at least partially coated in a substance which is substantially reflective to ultraviolet irradiation.

This is particularly advantageous in that such coatings are easily and quickly applied, yielding a reflective layer that is consistent in thickness and reflectivity. This also enables the fabrication of the irradiation chamber in a material that is substantially transparent to ultraviolet light (e.g. glass), the coating being removed from or otherwise not disposed thereupon at the locations where the UV-LEDs project into the irradiation chamber. The construction of the irradiation chamber may thereby be made much more inexpensive, simple, and resistant to leakage.

In a preferred embodiment, the plurality of UV-LEDs are disposed upon an exterior surface of the irradiation chamber.

This is advantageous in that the UV-LEDs are disposed completely outside of the flow of liquid through the irradiation chamber, and there are no openings or other discontinuities in the irradiation chamber aside from any inlet(s) and outlet(s). Furthermore, the disposition of the UV-LEDs on an exterior surface of the irradiation chamber simplifies the positioning of their electrical supply wiring, and facilitates any maintenance that may need to be performed on the UV-LEDs.

According to a second aspect, the invention is directed towards a beverage dispensing apparatus comprising an apparatus for purifying liquid as described above.

Such a beverage dispensing apparatus is advantageous in that it realizes in a practical application the advantages of the liquid purifying apparatus as described above.

### BRIEF DESCRIPTION OF THE FIGURES

FIGS. 1A and 1B are respectively longitudinal and lateral section views of an apparatus for purifying liquid, according to a first embodiment;

FIGS. 2A and 2B are respectively a side view and a lateral section view of an apparatus for purifying water according to a second embodiment; and

FIGS. 3A and 3B are respectively a side view and a lateral section view of an apparatus for purifying water according to a third embodiment.

### DETAILED DESCRIPTION

For a complete understanding of the present invention and the advantages thereof, reference is made to the following detailed description of the invention.

It should be appreciated that various embodiments of the present invention can be combined with other embodiments of the invention and are merely illustrative of the specific ways to make and use the invention and do not limit the scope of the invention when taken into consideration with the claims and the following detailed description.

Further, this document describes groups of components, which are referenced with both a numeral and a letter, e.g. “widgets 10A, 10B, 10C . . . .” When such terminology is employed, it should be understood that the components in the group are substantially identical; that when the both the numeral and letter are used it should be understood as referencing individual members of the group, while when only the numeral is used it should be understood as referencing the group in its entirety.

As used in this specification, the words “comprises”, “comprising”, and similar words, are not to be interpreted in an exclusive or exhaustive sense. In other words, they are intended to mean “including, but not limited to.

Any reference to prior art documents in this specification is not to be considered an admission that such prior art is widely known or forms part of the common general knowledge in the field.

The invention is further described with reference to the following examples. It will be appreciated that the invention as claimed is not intended to be limited in any way by these examples.

The main principle of the invention is first described.

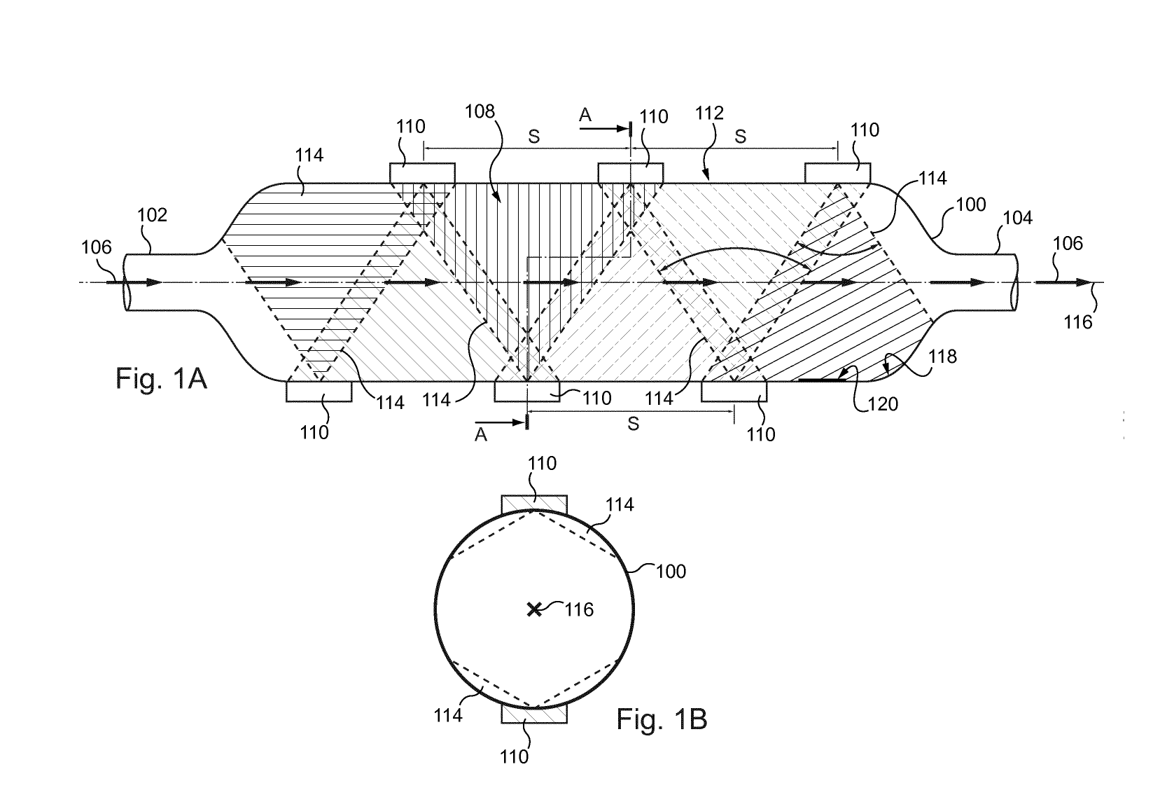

FIGS. 1A and 1B are respectively longitudinal and lateral section views of a device for purifying liquid. In FIG. 1A, the device is represented by the irradiation chamber 100, which is a substantially tubular, elongated structure having an inlet 102 and an outlet 104. The inlet 102 is adapted to receive a flow 106 of liquid, which is conducted through the cavity 108 of the irradiation chamber and out the outlet 104.

While the flow 106 is in the cavity 108 of the irradiation chamber 100, it is irradiated with ultraviolet light emitted by the UV-LEDs 110. The UV-LEDs are disposed upon an exterior surface 112 of the irradiation chamber 100, which is transparent to ultraviolet light where the UV-LEDs 110 are disposed. The In this way, the flow 106 of liquid is irradiated by each of the UV-LEDs 110 in turn, as it flows through the irradiation chamber 100.

Each of the UV-LEDs 110 emits ultraviolet light in a conical emission pattern 114, which has its point at the UV-LED 110 and gradually expands outwards as it propagates across the cavity 108 of the irradiation chamber 100. In FIG. 1A, each of the emission patterns 114 is given a different cross-hatching, to illustrate their overlapping nature.

In this embodiment, the UV-LEDs 110 are disposed along the irradiation chamber 100 with a consistent linear spacing, such that as one moves along the longitudinal axis 116 of the irradiation chamber 100, successive UV-LEDs 110 are separated by a distance 1/2s, with successive UV-LEDs 110 on one side being thus separated by a distance of s. The value of s is chosen as a function of the diameter of the irradiation chamber 110 and the angle of each emission cone 114 so that, as depicted here, each emission cone extends to an edge of at least one of the UV-LEDs 110 opposite. In this way, any dead zone around the UV-LEDs is minimized.

In most embodiments, it will be advantageous to ensure that the internal surface 118 of the irradiation chamber 100 is reflective to ultraviolet light. This will further serve to reduce, or even eliminate, any dead zones in the irradiation chamber 110, in that the portions of the volume of the irradiation chamber 100 which are not directly illuminated by one of the UV-LEDs 110 are irradiated by the reflected light.

In addition, this reflective property improves the sterilization efficiency of the irradiation chamber 100, in that UV light which does not irradiate a pathogenic microorganism directly can still do so after reflecting off of the interior surface 118 one or more times.

In practice, this reflective property can be achieved by the deposition of a coating 120 upon the interior surface 118 of the irradiation chamber 100, which is here only partially depicted in the interest of clarity. This coating can be, for example, a layer of a polymer such as polytetrafluoroethylene (PTFE), a metallic coating such as gold or silver, or some combination of these or other appropriate substances.

The means by which this coating is applied will depend on the particulars of the embodiment. For example, the irradiation chamber may be provided as transparent glass, and the coating applied by vapor deposition upon the internal surface of the chamber.

FIG. 1B offers further illustration of this, in the form of the section A-A at the section line shown in FIG. 1A. Here, it can be seen that a flow (not shown) of fluid passing through the irradiation chamber 100 will be irradiated throughout its section, as the UV-LEDs 110 illuminate the entirety of its circular cross-section. Thus, even when the dead zones cannot be totally eliminated from the irradiation chamber 100, the flow of the liquid through the chamber will ensure complete irradiation.

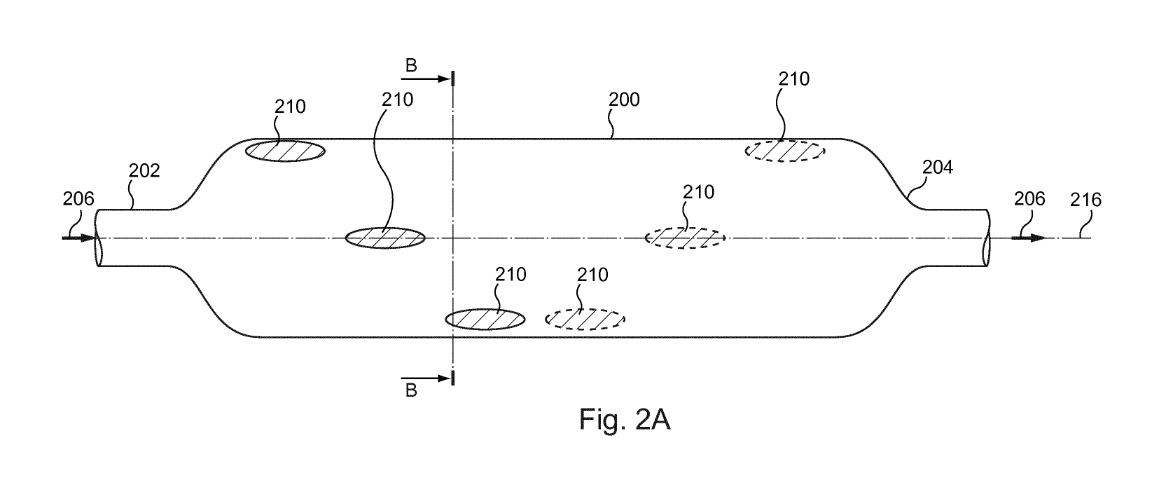

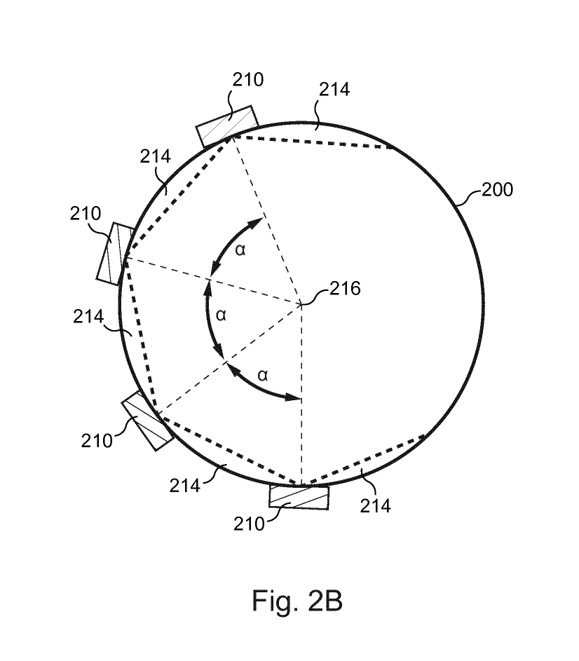

FIG. 2A is a side view of an apparatus for purifying water according to a second embodiment. In this embodiment, the irradiation chamber 200 is, as in the previous embodiment, provided with an inlet 202 and an outlet 204 adapted to conduct a flow 206 of liquid through the irradiation chamber 200. Ultraviolet radiation is projected into the cavity 208 of the irradiation chamber 200 by the UV-LEDs 210.

The UV-LEDs 210 are disposed upon the irradiation chamber 200 with a substantially uniform spacing both in a linear sense along the longitudinal axis 216, and in an axial sense about said longitudinal axis 216. The UV-LEDs 210 are thus arranged upon the irradiation chamber 200 in a helical arrangement that realizes the advantages described above.

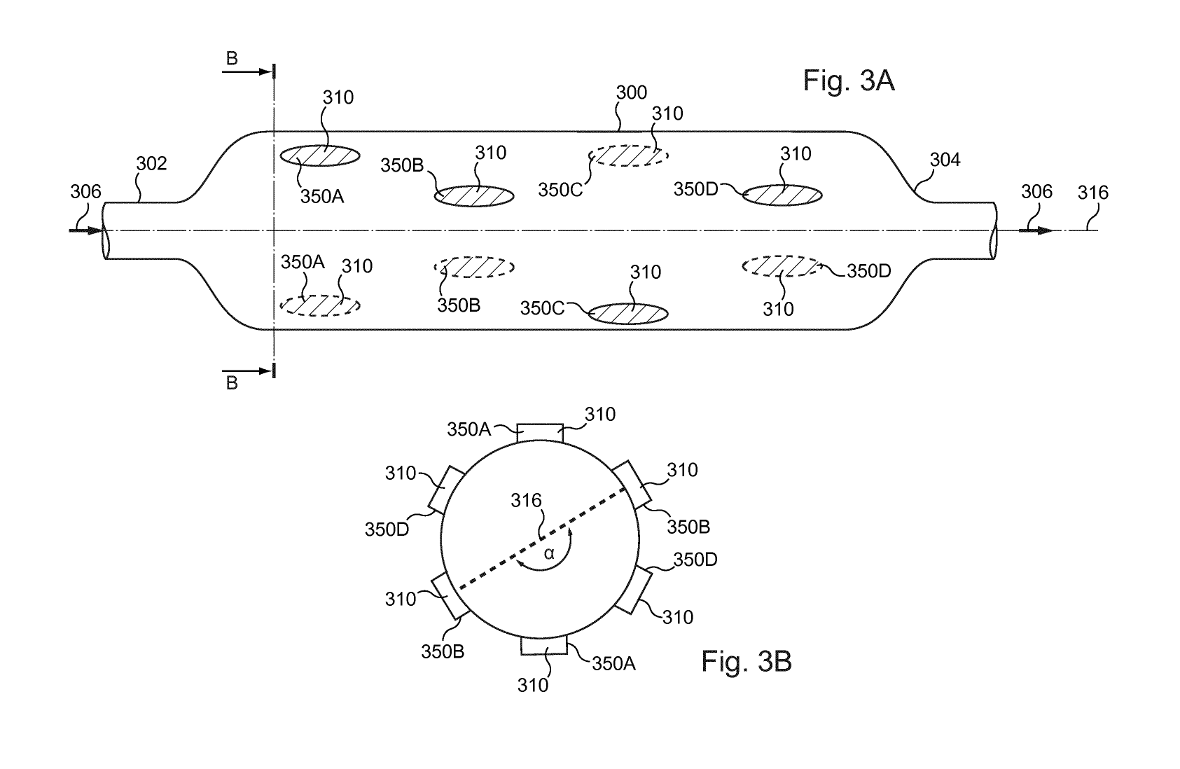

FIG. 2B is a lateral section view of the irradiation chamber 200 through the section line B-B depicted in FIG. 3A. In FIG. 2B, it is seen that the angular spacing of the UV-LEDs 210 about the longitudinal axis 216 (here designated by the symbol φ is substantially constant between each of the UV-LEDs 210. This Figure also shows the wide-angled conical emission patterns 214.

Thus, it can be seen that in any particular application, the propagation of the UV light within the irradiation chamber can be controlled by modifying the parameters thus far described, including the angle of the emission patterns, the longitudinal spacing between UV-LEDs, the angular spacing of the UV-LEDs, the total active length of the irradiation chamber, and the number of the UV-LEDs.

The user can thus adapt the apparatus to the particular needs of the application for which it is destined; for instance, an application where a high degree of sterilization is desired such as a dispenser for infant formula, can be provided with many UV-LEDs with tight linear and angular spacing, while other applications where the need for sterilization is not so acute may be provided with fewer UV-LEDs and wider spacing.

FIGS. 3A and 3B are respectively a side view and lateral section view of a liquid purifying apparatus according to a third embodiment. As in the previous two embodiments discussed above, the apparatus comprises an irradiation chamber 300, provided with an inlet 302 and an outlet 304 configured to direct a flow 306 of liquid through the cavity 308 of the irradiation chamber 300. The irradiation chamber is further provided with a plurality of UV-LEDs 310, which project into the irradiation chamber 310 as in the two embodiments discussed above.

In this embodiment, the UV-LEDs 310 are arranged in pairs 350A, 350B, 350C, and 350D. Each pair 350 is disposed so that the two UV-LEDs project upon each other, such that they are at the same linear position with respect to the longitudinal axis 316, but have a 180° angular separation about said longitudinal axis 316. The UV-LEDs 310 in each of the pairs 350 will thereby mutually illuminate each other, eliminating any dead zone around them.

Furthermore, the pairs 350 of UV-LEDs 310 are disposed along the length of the irradiation chamber 300 with a substantially constant linear spacing, and about the longitudinal axis 316 of the irradiation chamber 300 with a substantially constant axial spacing, substantially as described in relation to the two previous embodiments. This spacing ensures that the UV-LEDs 310 of each pair 350 are also illuminated by at least one UV-LED 310 of another pair 350, in the same way as described above.

In this way, a thorough purification of the flow 306 of liquid is achieved. Moreover, because each UV-LED 310 is illuminated both by its complement UV-LED 310 in its own pair 350, and by a UV-LED 310 in another one of the pairs 350, there is achieved a redundancy should one of the UV-LEDs 310 fail. In this way, the reliability of the system is improved.

FIG. 3B is a lateral section view of the irradiation chamber 300, taken at the section line C-C as depicted in FIG. 3A. Here, it can be seen that the angular separation a between the UV-LEDs 310 of the pair 350B is 180°; i.e. that the UV-LEDs 310 of the pair 350B are diametrically opposed from one another. While not depicted for clarity, this angular relation is the same for each of the other pairs 350A, 350D of UV-LEDs 310.

Although the invention has been described by way of example, it should be appreciated that variations and modifications may be made without departing from the scope of the invention as defined in the claims. Furthermore, where known equivalents exist to specific features, such equivalents are incorporated as if specifically referred in this specification.

In a general sense, elements described in the foregoing disclosure should not be taken as being limited to the combinations and configurations described in the foregoing example embodiments. Recombination of the elements described above according to the particulars of each application should be considered as envisioned when not in direct contradiction to this disclosure.

In particular, it should be recognized that, while the above embodiments describe embodiments where there is a constant flow of liquid through the irradiation chamber, the invention is equally directed towards embodiments where said flow is not constant, i.e. so-called “static” reactors. In such an embodiment, it may be that a volume of liquid flows into the irradiation chamber, is irradiated, and then subsequently flows out. The foregoing disclosure should not, therefore, be construed as being limited to constant-flow apparatuses such as the embodiments discussed above.

Also, while it is envisioned that an apparatus according to the present invention be integrated into a beverage dispensing apparatus, it may equally be possible to employ such an apparatus in other applications, for example in commercial, industrial, medical, or other such applications where reliable purification of a liquid is sought. In particular, it may be advantageous to incorporate such an apparatus into devices such as beverage vending machines, coffee or tea dispensers, or dispensers for prepared food such as soups, cereals, infant formula, or the like.

It should thus be understood that various changes and modifications to the presently preferred embodiments described herein will be apparent to those skilled in the art. Such changes and modifications can be made without departing from the spirit and scope of the present invention and without diminishing its attendant advantages. It is therefore intended that the appended claims be considered as including any embodiment which is derived at least partially from it.

1. An apparatus for purifying liquid, comprising a substantially tubular irradiation chamber adapted to conduct a flow of liquid therethrough, and a plurality of UV-LEDs disposed upon and configured to project ultraviolet radiation into the irradiation chamber and thereby irradiate the flow of liquid, characterized in that the plurality of UV-LEDs are configured such that each of the UV-LEDs is directly illuminated by the ultraviolet irradiation emitted by at least one other of the UV-LEDs.

2. The apparatus of claim 1, wherein the plurality of UV-LEDs are distributed along a length of the irradiation chamber with a substantially uniform linear spacing.

3. The apparatus of claim 1, wherein the plurality of UV-LEDs are distributed along the perimeter of the irradiation chamber with a substantially uniform angular spacing about a longitudinal axis of the irradiation chamber.

4. The apparatus of claim 1, wherein each of the UV-LEDs is disposed upon the irradiation chamber directly opposite another of the UV-LEDs, thereby defining a plurality of UV-LED pairs.

5. The apparatus of claim 4, wherein the UV-LED pairs are distributed along a length of the irradiation chamber with a substantially uniform linear spacing, and along the perimeter of the irradiation chamber with a substantially uniform angular spacing about a longitudinal axis of the irradiation chamber.

6. The apparatus of claim 1, wherein the distance along a wall of the irradiation chamber between any two adjacent UV-LEDs is less than or equal to twice a width of the irradiation chamber multiplied by the tangent of one-half the angle of emission of the UV-LEDs.

7. The apparatus of claim 1, wherein the irradiation chamber has a substantially constant cross-section.

8. The apparatus of claim 7, wherein the irradiation chamber has a substantially circular cross-section.

9. The apparatus of claim 1, wherein the UV-LEDs have an angle of emission equal to or greater than 90°.

10. The apparatus of claim 9, wherein the UV-LEDs have an angle of emission between 110° and 130° inclusive.

11. The apparatus of claim 1, wherein at least part of an interior surface of the irradiation chamber is substantially reflective to ultraviolet radiation.

12. The apparatus of claim 1, wherein the interior surface of the irradiation chamber is at least partially coated in a substance which is substantially reflective to ultraviolet irradiation.

13. The apparatus of claim 1, wherein the plurality of UV-LEDs are disposed upon an exterior surface of the irradiation chamber.

14. A beverage dispensing apparatus comprising an apparatus for purifying liquid comprising a substantially tubular irradiation chamber adapted to conduct a flow of liquid therethrough, and a plurality of UV-LEDs disposed upon and configured to project ultraviolet radiation into the irradiation chamber and thereby irradiate the flow of liquid, the plurality of UV-LEDs are configured such that each of the UV-LEDs is directly illuminated by the ultraviolet irradiation emitted by at least one other of the UV-LEDs.
